(19) United States
(12) Patent Application Publication
(10) Pub. No.: US 2013/0225956 A1
(43) Pub. Date: Aug. 29, 2013

(54) Transdermal Sensor

(71) Applicant: National Taipei University of Technology, (US)

(72) Inventors: Jung-Tang Huang, Taipei (TW); Kuo-Yuan Hwa, Taipei (TW); Chih-Cheng Lu, Taipei (TW)

(73) Assignee: National Taipei University of Technology, Taipei (TW)

(21) Appl. No.: 13/625,460

(22) Filed: Sep. 24, 2012

(30) Foreign Application Priority Data
Sep. 23, 2011 (TW) 100217917

(51) Int. Cl. A61B 5/053

(52) U.S. Cl. CPC A61B 5/0537
USPC 600/345

(57) ABSTRACT

The present invention provides a transdermal sensor for detecting a concentration of a hypodermal target molecule, comprising: a substrate; a plurality of microneedles fixed on said substrate; a signal processing unit, which is electrically connected to said microneedles; and a power supply unit for providing the working power. The transdermal sensor of the present invention detects long-term, real-time concentration of a hypodermal target molecule for a doctor to evaluate a physiological status of an user with minimal invasive piercing and pain.

This application claims benefit under 35 U.S.C 119(a) of Taiwan Utility Model Patent Application No. 100217917, filed Sep. 23, 2011, the entire content of which is incorporated by reference herein.

### TECHNOLOGY FIELD

The present invention relates to a transdermal sensor, particularly a transdermal sensor for long-term, real-time measurement/monitoring of the concentrations of hypodermic target molecules to obtain physiological signals of the human body.

### BACKGROUND OF THE INVENTION

Interstitial fluid (or tissue fluid) consists of a water solvent containing amino acids, sugars, fatty acids, coenzymes, hormones, neurotransmitters, salts, and waste products from the cells, and its composition depends upon the exchanges between the cells in the biological tissue and the blood. Subcutaneous interstitial fluid is a preferred site for target molecule sensing, as it is easily accessed and carries a lower risk of infection than the blood stream.

When a pharmaceutical is administered to a subject, it will be slowly released into the interstitial fluid for a long period of time. During the clinical trial phases of drug development, continuous monitoring of the concentration of the drug in the interstitial fluid is usually required. In addition, it is very common in a medical procedure to sample interstitial fluid and perform examination or analysis.

Presently commercially available physiological examination equipments or methods used by medical personnel for sampling interstitial fluid involve piercing the cuticle with a needle to draw interstitial fluid for analytical examination. However, such sampling methods are painful and may cause infection. Various types of microneedle arrays for stamping have been developed, and some are commercially available (Donnelly RF et al., Drug Deliv 2010; 17:187-207, and Gomaa YA et al., Toxicol In Vitro 2010; 24:1971-1978). However, their main use is in transdermal drug delivery. Some microneedle arrays, including hollow microneedles for direct ISF sampling, are used to pretreat skin before sampling, but there are not sufficient data on their practical application for ISF extraction (Wang PM et al., Diabetes Technol Ther 2005; 7:131-141).

Furthermore, for blood glucose tracking or monitoring of drug concentration, which require long-term and continuous measuring, the multiple daily measurements are a torture to the patients. Further, current examination equipments measure “off-line” concentrations of target molecules, that is, the measurement is performed after the interstitial fluid is drawn from the human body. And if the concentration of the molecule to be measured in the interstitial fluid is extremely low, it is necessary to draw more interstitial fluid for accurate measurement.

Therefore, it is desirable to develop a more efficient device or method for measuring the concentration of a target molecule in the interstitial fluid.

### BRIEF SUMMARY OF THE INVENTION

The present invention relates to a transdermal sensor which utilizes an array of microneedles to pierce skin. The minimally invasive piercing effectively reduces the pain of the users and simultaneously achieves the goal of sampling tissue fluid.

The present invention provides a transdermal sensor which continuously monitors the concentration of hypodermal target molecules by microneedles. The sensitivity of the transdermal sensor is increased through accumulating a trace amount of target molecules at the electrode.

The transdermal sensor of the present invention is able to transmit measured signals outward and to receive instructing signals from a doctor.

The present invention provides a transdermal sensor for detecting the concentration of hypodermal target molecules, comprising: a substrate; a plurality of microneedles fixed on said substrate; a signal processing unit, which is electrically connected to said microneedles; and a power supply unit for providing the working power.

The following improvements can be achieved by the present invention:
1. The discomfort and pain caused by sampling are reduced and thus the willingness of the user is increased.
2. The concentration of hypodermal target molecules can be monitored continuously in a long term with minimal invasiveness, and thus skin wounds and infections are reduced.
3. Sensitivity of the transdermal sensor is enhanced through long-term detection and accumulation of the target molecules at the microneedles without drawing a large amount of tissue fluid.
4. User's physiological signals can be periodically monitored and subjected to manual diagnosis, and then an instructing signal may be sent to the user for reminding of medication or of paying attention to his or her physiological status.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1A is a three-dimensional illustration of an embodiment of the transdermal sensor of the present invention. FIG. 1B is a top view and FIG. 1C is a side view of the embodiment.

FIG. 2A is a three-dimensional illustration of another embodiment of the transdermal sensor of the present invention. FIG. 2B is a side view of said embodiment.

FIGS. 3A-3L illustrate the manufacturing process of the microneedles. FIG. 3A shows a microneedle substrate and the photoresist pattern is defined as shown in FIG. 3B. FIG. 3C shows the microneedle substrate after isotropic etching, and FIG. 3D shows the etched microneedle substrate with photoresist removed. FIG. 3E illustrates the deposition of a gold layer on the microneedles. FIGS. 3F-3H show a way to define a reference electrode microneedle, i.e. by lithography of platinum electroplating with a resist covering the other microneedles. FIGS. 3I-L show the assembly of the microneedles onto a sensor substrate. FIG. 3I shows the coating of a polymer material, and FIG. 3J illustrates the polish of the reverse side (relative to the microneedle side) of the microneedle substrate. FIGS. 3K-L illustrate the system-in-package assembly of the microneedles onto a sensor substrate.

FIG. 4A and 4B show two embodiments of the present invention respectively, illustrating the real-time continuous glucose monitoring systems according to the invention.

FIGS. 5A and 5B show the examples of peak 1 and peak 2 of the current measured by cyclic voltammetry, respectively. FIG. 5C shows the linearity response of peak 1 current value from the 10-day long-acting GOx-coated microneedles of the present invention. FIG. 5D shows the linearity response of peak 2 current value from the 10-day long-acting GOx-coated microneedles of the present invention.

### DETAILED DESCRIPTION OF THE INVENTION

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

The present invention is further illustrated by the following descriptions and drawings, which are intended for mere demonstration and explanation but not limitation of the present invention to specific forms. The present invention envisions other variations in addition to those described herein. It is believed that those skilled in the art can achieve the whole scope of the present invention based on the descriptions herein.

The present invention provides a transdermal sensor for detecting a concentration of a hypodermal target molecule, comprising: a substrate; a plurality of microneedles fixed on said substrate; a signal processing unit, which is electrically connected to said microneedles; and a power supply unit for providing the working power.

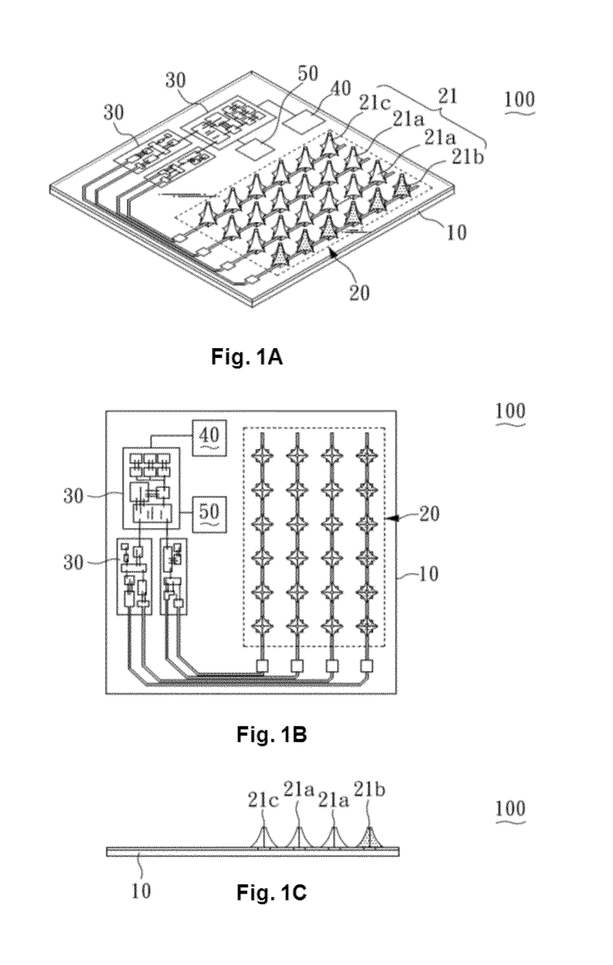

As shown in FIG. 1A, in one embodiment of the present invention, the transdermal sensor 100 for detecting a concentration of a hypodermal target molecule comprises a substrate 10, a plurality of microneedles 20, a signal processing unit 30, and a power supply unit 40. The target molecule may be a biological molecule, such as glucose, cortisol, or fatty acids. The target molecule may also be a pharmaceutical molecule. The transdermal sensor of the present invention may be used for pharmaceutical monitoring during the administration of a medication for a chronic disease or a specific pharmaceutical. Personalized medication of a specific dosage or frequency of administration can be provided based on the individual metabolism of the pharmaceutical.

According to the present invention, the substrate 10 may be a printed circuit board or a chip. Stainless steel, having high strength and being biocompatible, has been widely used in implants for the human body for years. In one embodiment of the present invention, the substrate is made of stainless steel.

According to the present invention, the plurality of microneedles 20 may be an array of microneedles. The array may be linear, rectangular, circular, or multicircular. When the transdermal sensor 100 is adhered to the skin surface, the microneedle 21 pierces the skin surface and detects the concentration of a hypodermal target molecule. Since the microneedle 21 is stuck in the skin surface for an extended period, high biocompatibility is required. Specifically, the material of the microneedle may be selected from the group consisting of stainless steel, nickel, nickel alloy, carbon nanotube, or silicon. In addition, biocompatible metals such as gold, platinum, palladium, nickel, or an alloy thereof may be deposited on the microneedle 21 to increase biocompatibility. In one embodiment of the present invention, the microneedle is made of stainless steel and deposited with metal gold.

The microneedle of the present invention has a diameter of less than 100 μm, preferably less than 50 μm, and a length between 50 and 3,000 μm. Such design can reduce the pain caused by the piercing and improve patient cooperativeness. According to the present invention, the spacing/distance between adjacent microneedles may be between 50 and 1,000 μm (from tip to tip).

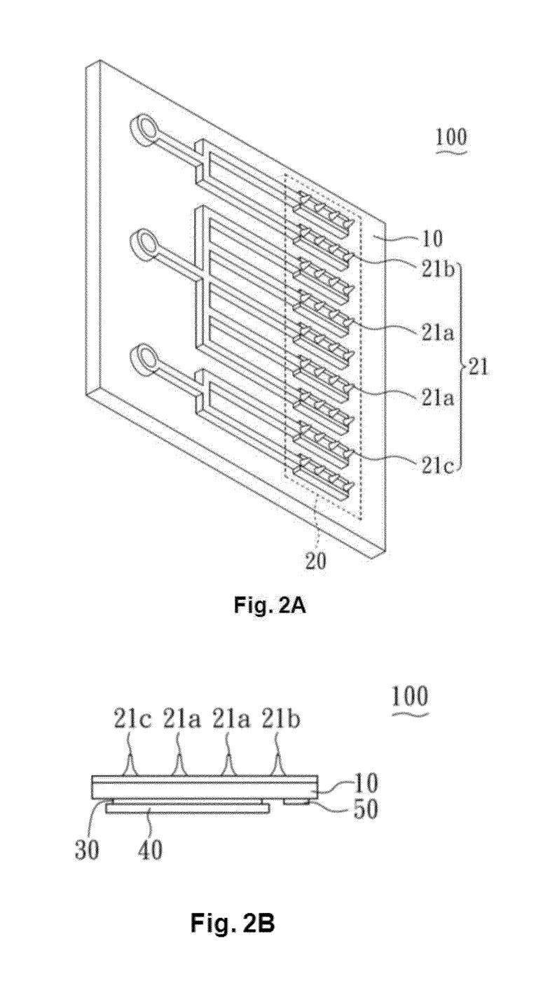

As shown in FIGS. 1B and 2A, the plurality of microneedles 20 are arranged on the substrate 10 in the form of an array and are electrically connected to the substrate 10 for detecting the concentration of a target molecule through an electrochemical method, such as electrochemical impedance (EIS), cyclic voltammetry, or amperometry. According to the present invention, the plurality of microneedles may be fixed on the substrate individually (as shown in FIG. 1B), or may be group in row needles and fixed on the substrate (as shown in FIG. 2A).

In one embodiment of the present invention, the electrochemical impedance method is employed (see FIGS. 1C and 2B). First, the working electrode(s), reference electrode(s), and counter electrode(s) are to be defined. Among the plurality of microneedles 20, define at least one microneedle as working electrode(s) 21a, at least one microneedle as reference electrode(s) 21b, and at least one microneedle as counter electrode(s) 21c. Based on the high impedance characteristic of the target molecule, impedance values are continuously detected to evaluate the concentration of the target molecules. In addition, sensitivity of the transdermal sensor 100 can be enhanced through the accumulation of a trace amount of target molecules at the electrode.

For specificity, the microneedles (for example, the working electrode(s), microneedle(s) 21a) may be subjected to surface modification, in view of the target molecule to be detected. Specifically, a molecule selected from the group consisting of an enzyme, an antibody, an aptamer, a single-chain variable fragment (ScFv), a carbohydrate, and a combination thereof, may be coated on the surface of the microneedles. In one embodiment of the present invention, the working electrodes are modified with glucose oxidase (GOx) for (blood) glucose detection. For coupling of an antibody, self-assembled monolayer (SAM) may be applied to the microneedles deposited with gold, before adding the antibody. To increase sensitivity, carbon nanotubes may be further mixed into the surface gold layer.

As shown in FIGS. 1B and 2B, the transdermal sensor 100 comprises a signal processing unit 30 and a power supply unit 40, and may further comprises a wireless transmission unit 50. The aforementioned units may be fixed on the same side with the plurality of microneedles 20 on the substrate 10 to form a thin transdermal sensor, or they may be fixed on the opposite side on the substrate 10 for miniature design.

According to the present invention, the signal processing unit is electrically connected to the microneedles so as to receive an electrical signal in connection with the target molecule concentration. According to related technologies known in the art, the electrical signal may be amplified and converted to a digital signal, and the digital signal may be further processed based on an algorithm and transformed into a sensor signal that reflects a physiological status of the user.

The transdermal sensor of the present invention may be in the form of a patch and used for long-term, real-time detection and monitoring. Thus, in some embodiments of the present invention, the power supply unit 40 may provide working power for the transdermal sensor for a long term, for example, for more than 10 days, preferably more than 20 days, and more preferably more than 30 days.

According to the present invention, the wireless transmission unit is electrically connected to the signal processing unit, and may transmit the sensor signal received from the signal processing unit to a doctor for further review and diagnosis. If the doctor considers that immediate treatment or medication is required, he or she may then send an instruction signal to the user. The wireless transmission unit would receive the instruction signal and the transdermal sensor may provide a signal to remind the user to pay attention to his or her physiological status or to take medication.

Further, when simultaneous detection of multiple pharmaceutical or biological molecules is required, multiple transdermal sensors may be combined and packaged into a system in package (SIP). In principle, the processing circuit of the transdermal sensor may be manufactured separately from the microneedles to simplify the manufacturing process. An example of the manufacturing process for the microneedles is described in detail as below.

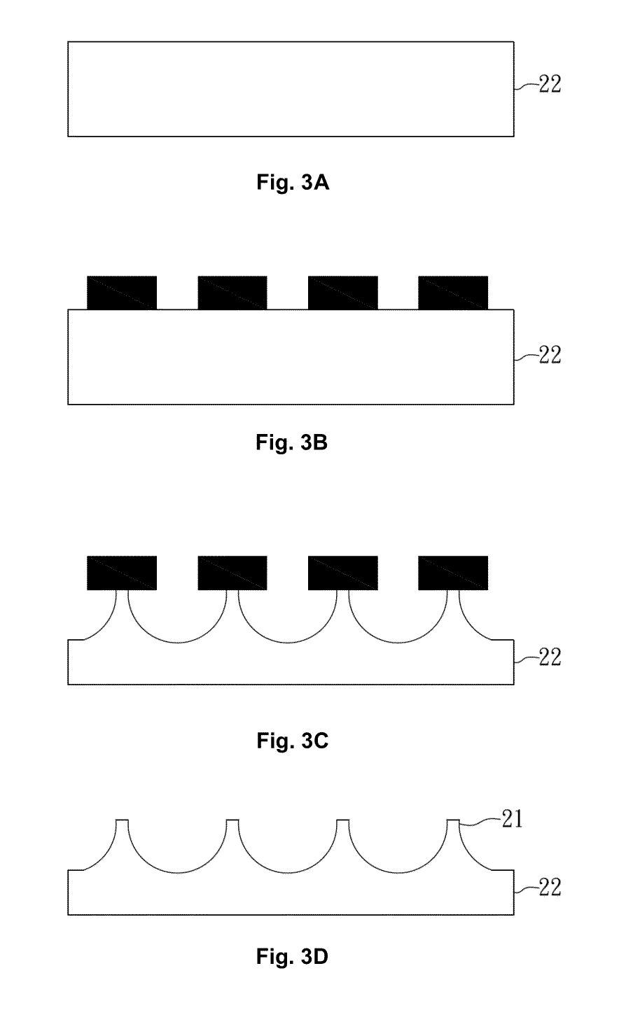

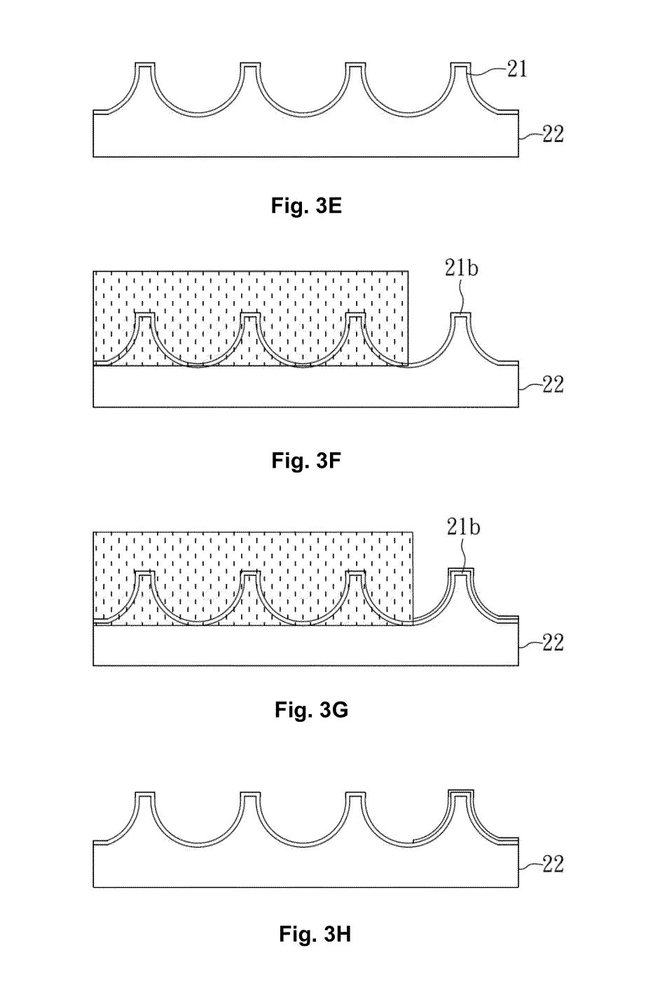

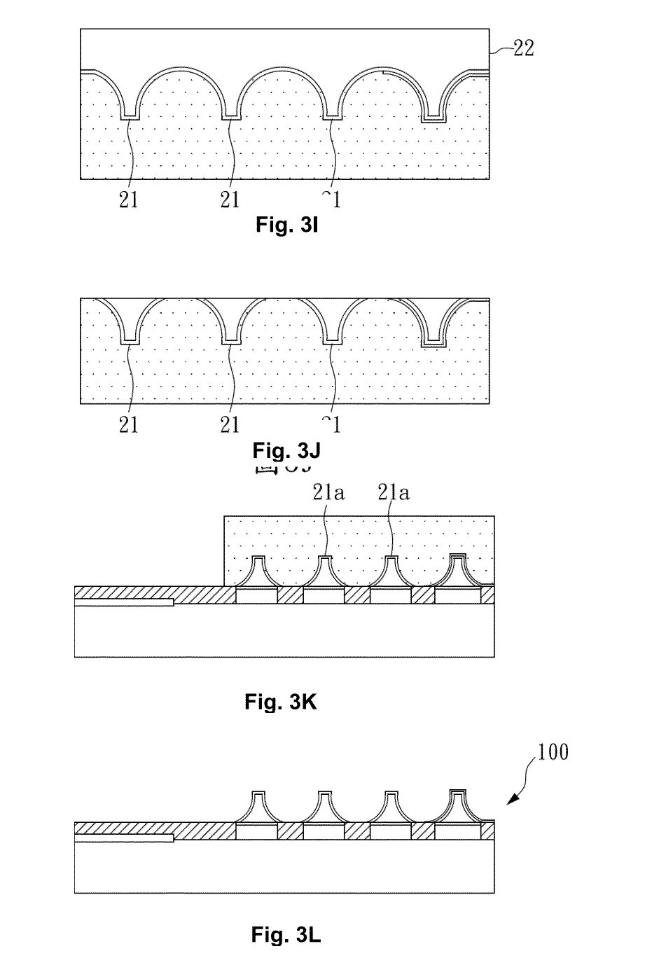

FIGS. 3A to 3L illustrate the manufacturing process of the microneedles. First, as shown in FIG. 3A, a microneedle substrate 22 made of stainless steel is provided. Second, define the photoresist pattern based on photolithography technique, as shown in FIG. 3B. Then, perform isotropic etching (e.g., an electrochemical etching) to shape the microneedles, as shown in FIG. 3C. Finally, as shown in FIG. 3D, the photoresist is removed to obtain the shaped microneedles.

As shown in FIG. 3E, gold may be further deposited on the microneedles. A layer of gold was deposited on the surface of the microneedle 21 by electroplating. In addition, a microneedle 21b may be defined as a reference electrode as shown in FIGS. 3F-3H. The microneedle 21b is defined as a reference electrode by lithography of platinum electroplating with a resist covering the other microneedles. FIGS. 3K-L illustrate the system-in-package assembly of the microneedles onto a sensor substrate. The microneedles 21 are coated with polymer material, as shown in FIG. 3I, and the reverse side (relative to the microneedle side) of the microneedle substrate 22 is polished to separate the microneedles, as shown in FIG. 3J. Subsequently, define a microneedle 21a as a working electrode, and modify the working electrode 21a by lithography with a resist covering the other microneedles. Finally, assemble the microneedles 21 onto the substrate 10 using system-in-package technology, as shown in FIGS. 3K-L.

Take cortisol detection as an example. Cortisol is the most abundant steroid in blood and involves in the functions of anti-inflammation, maintaining blood pressure, gluconeogenesis, calcium absorption, and secretion of gastric juices. Cortisol may serve as the basis for diagnosing Addison's disease, Cushing's syndrome, hypopituitarism, congenital adrenal hyperplasia, and cancer. For tracking changes of in vivo cortisol concentration along time, chimeric monoclonal antibodies (cMAb) may be covalently immobilized on the microneedles of the transdermal sensor of the present invention.

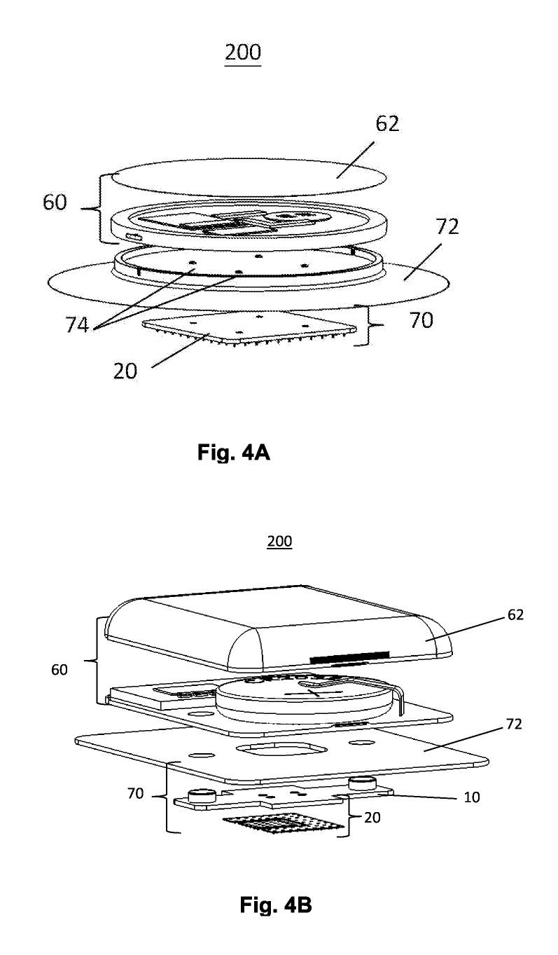

FIGS. 4A and 4B illustrate another embodiment of the present invention, a real-time continuous glucose monitoring system 200, comprising a control and wireless transmission module 60, and a sensor module 70. The control and wireless transmission module 60 comprises a cap 62, and a PCB to carry a signal processing unit, a power supply unit, and a wireless transmission unit. The sensor module 70 in FIG. 4A comprises a plurality of microneedles 20, a ring tape 72, and a plurality of electrical contacts 74. The sensor module 70 in FIG. 4B comprises a plurality of microneedles 20, a tape 72, and a substrate 10.

The present invention is further illustrated by the following examples, which are provided for the purpose of demonstration rather than limitation.

### Example 1

### Functionalizing Microneedles with Glucose Oxidase (GOx) and/or Hydroxybutyrate Dehydrogenase (HBHD) on the Surface

### Materials and Methods

The gold surface of the electrode was first modified with 3-mercaptopropionic acid (3-MPA) to form a self-assembled monolayer. And then the electrode was soaked in an aqueous solution containing a selected enzyme (GOx or HBHD) and a matrix solution. The enzyme solution was prepared with GOx or the other selected enzyme with matrix solution in 0.1 M PBS (pH 7.0). The electrode was immersed in the enzyme solution for 24 hours at 4° C. The excess amount of enzymes was washed away with 0.1 M PBS (pH 7.0). Prepare a glucose solution in the concentration between 3.6-5.8 mM (human blood glucose level) and a PBS as blank solution. Immerse the enzyme immobilized gold electrode in to the glucose solution and PBS, and measure the current by cyclic voltammetry.

### 1.2 Results

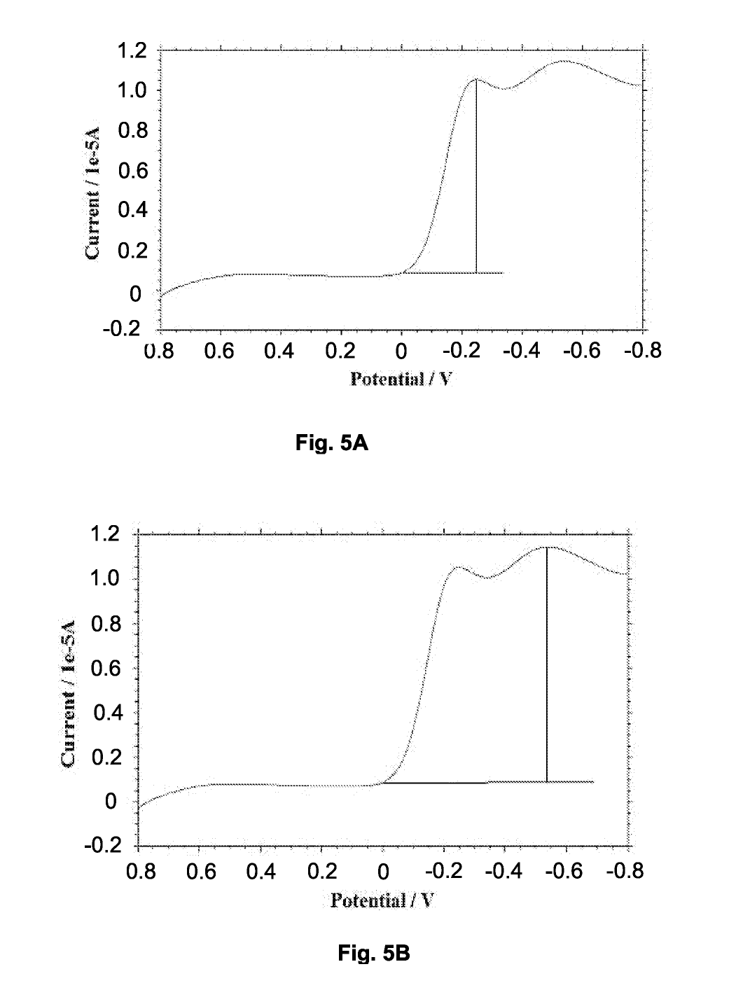

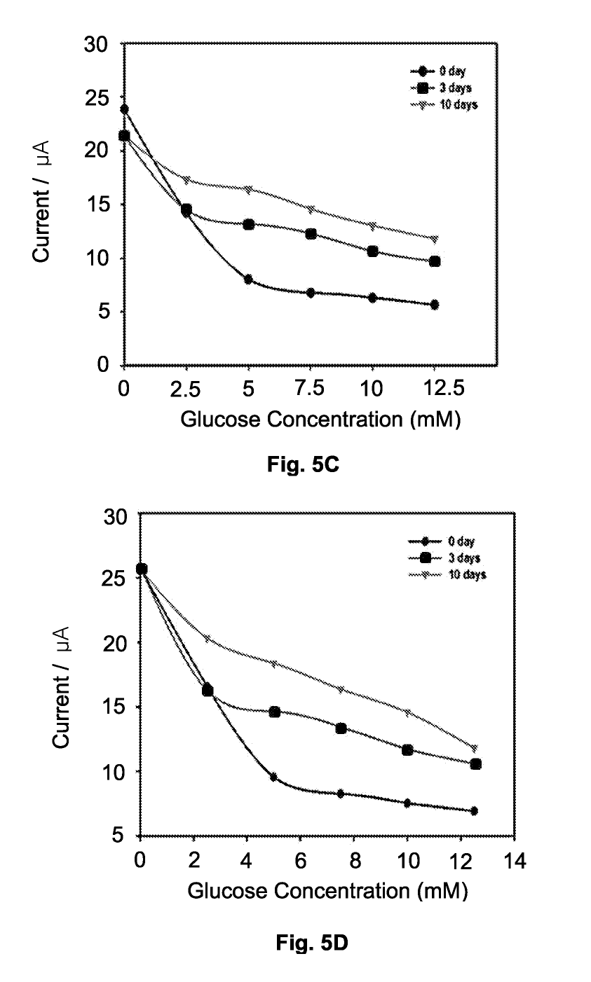

As shown in FIGS. 5A to 5D, the voltage responses depend on the concentration of glucose in the test solution. The enzymatic activity of GOx maintains for up to 10 days. Linearity response from the 10-day long-acting GOx-coated electrode was observed. Moreover, in peak 1, the R² values were found to be 0.7608 in day 1, 0.8225 in day 3, and 0.9491 in day 10, respectively. In peak 2, the R² values were found to be 0.7814 in day 1, 0.7853 in day 3, and 0.9574 in day 10, respectively. See table 1 below.

TABLE 1
The correlation coefficient values of peak 1 and peak 2.

| | Peak 1 | Peak 2 |
|---|---|---|
| Day 1 | R² = 0.7608 | R² = 0.7814 |
| Day 3 | R² = 0.8225 | R² = 0.7853 |
| Day 10 | R² = 0.9574 | R² = 0.9574 |

The above-disclosed preferred embodiments of the present invention are not intended as limitations to the present invention. Those skilled in the art of the present invention should be able to make changes and modifications within the spirit and scope of the present invention, and such changes and modifications would fall within the protected scope of the present invention as defined by the appended claims.

What is claimed is:

1. A transdermal sensor for detecting a concentration of a hypodermal target molecule, comprising:
a substrate;
a plurality of microneedles fixed on said substrate for an electrochemical detection;
a signal processing unit, which is electrically connected to said microneedles for receiving an electric signal of the electrochemical detection, and processing and transforming it into a sensor signal; and
a power supply unit, which provides working power.

2. The transdermal sensor of claim 1, further comprising a wireless transmission unit which is electrically connected to the signal processing unit for receiving and transmitting the sensor signal outward and for receiving an instructing signal.

3. The transdermal sensor of claim 2, wherein the electrochemical detection is based on electrochemical impedance (EIS), cyclic voltammetry, or amperometry.

4. The transdermal sensor of claim 3, wherein the plurality of microneedles comprise at least one microneedle serving as a working electrode, at least one microneedle serving as a reference electrode, and at least one microneedle serving as a counter electrode.

5. The transdermal sensor of claim 4, wherein the at least one microneedle serving as a working electrode is surface-modified.

6. The transdermal sensor of claim 5, wherein the at least one microneedle serving as a working electrode is modified by a molecule selected from the group consisting of an enzyme, an antibody, an aptamer, a single-chain variable fragment (ScFv), a carbohydrate, and a combination thereof.

7. The transdermal sensor of claim 6, wherein the enzyme is glucose oxidase (GOx) and/or hydroxybutyrate dehydrogenase (HBHD).

8. The transdermal sensor of claim 1, wherein the distance between adjacent microneedles, from tip to tip, is 50 to 1000 μm.

9. The transdermal sensor of claim 1, wherein the length of the microneedles is 50 to 3000 μm.

10. The transdermal sensor of claim 1, wherein the target molecule is a bio-molecule or a pharmaceutical molecule.

11. The transdermal sensor of claim 1, wherein the bio-molecule is glucose, cortisol, or fatty acids.

12. The transdermal sensor of claim 1, wherein the substrate is a printed circuit board or a chip.

13. The transdermal sensor of claim 1, wherein the microneedles are made of a material selected from the group consisting of stainless steel, nickel, nickel alloy, carbon nanotubes, and silicon.

14. The transdermal sensor of claim 1, wherein the e microneedles are deposited with metal gold.
